(19) United States
(12) Patent Application Publication
(10) Pub. No.: US 2020/0111765 A1
(43) Pub. Date: Apr. 9, 2020

(54) ELECTRONIC ASSEMBLY AND METHOD OF FORMING SAME

(71) Applicant: Medtronic, Inc., Minneapolis, MN (US)

(72) Inventors: Chunho KIM, Phoenix, AZ (US); Mark E. HENSCHEL, Phoenix, AZ (US); Yongqian WANG, Chandler, AZ (US); Shawn SHI, Tempe, AZ (US)

(21) Appl. No.: 16/594,577

(22) Filed: Oct. 7, 2019

Related U.S. Application Data
(60) Provisional application No. 62/743,124, filed on Oct. 9, 2018.

(51) Int. Cl. H01L 25/065; H01L 23/00; H01L 25/00; H01L 23/31

(52) U.S. Cl. CPC H01L 25/0657; H01L 24/06; H01L 24/03; H01L 23/315; H01L 25/50

(57) ABSTRACT

Various embodiments of an electronic assembly and a method of forming such assembly are disclosed. The electronic assembly includes a first integrated circuit package electrically connected to a second integrated circuit package. The first integrated circuit package includes a dielectric layer, a patterned conductive layer disposed within the dielectric layer, a device disposed on the first major surface of the dielectric layer and electrically connected to the patterned conductive layer, and an encapsulant layer disposed on the device and at least a portion of the first major surface of the dielectric layer. A conductive pillar of the second integrated circuit package is disposed within a trench of the first integrated circuit package such that the conductive pillar is electrically connected to a conductor disposed within the trench of the first integrated circuit package. The conductive pillar is electrically connected to a patterned conductive layer of the second integrated circuit package.

### CROSS-REFERENCE TO RELATED APPLICATION

This application claims the benefit of U.S. Provisional Application Ser. No. 62/743,124, filed Oct. 9, 2018, the entire content of which is incorporated herein by reference.

### TECHNICAL FIELD

This disclosure generally relates to electronic assemblies and methods of forming such assemblies.

### BACKGROUND

A wide variety of electronic assemblies such as those that are utilized for implantable medical devices (IMDs) employ electronic circuitry, e.g., for providing electrical stimulation of body tissue and/or monitoring a physiologic condition. Such IMDs may deliver electrical therapy energy in the form of shocking energy and stimulating pulses to selected body tissue and typically include output circuitry for generating the electrical energy under prescribed conditions and at least one lead bearing a stimulation electrode for delivering the electrical energy to the selected tissue. For example, cardiac pacemakers and implantable cardioverter-defibrillators (ICDs) have been developed for maintaining a desired heart rate during episodes of bradycardia or for applying cardioversion or defibrillation therapies to the heart upon detection of serious arrhythmias. Other nerve, brain, muscle, and organ tissue stimulating medical devices are also known for treating a variety of conditions.

Currently available IMDs, including ICDs and implantable pulse generators (IPGs), are typically formed having a metallic housing that is hermetically sealed and, therefore, is impervious to body fluids, and a header or connector assembly mounted to the housing for making electrical and mechanical connection with one or more leads. Such devices also possess telemetry capabilities for communicating with external devices. Over the past 20 years, IMDs have evolved from relatively bulky devices to complex miniaturized devices that exhibit increasing functionality. For example, numerous improvements have been made in cardioversion/defibrillation leads and electrodes that have enabled the cardioversion/defibrillation energy to be precisely delivered to selected one or more portions of upper and lower heart chambers and thereby dramatically reducing the delivered shock energy required to cardiovert or defibrillate the heart chamber. The high voltage output circuitry has also been improved in many respects to provide monophasic, biphasic, or multi-phase cardioversion/defibrillation shock or pulse waveforms that are efficacious, sometimes with particular combinations of cardioversion/defibrillation electrodes, in lowering the required shock energy to cardiovert or defibrillate the heart.

The miniaturization of IMDs is driving size and cost reduction of all IMD components, including the electronic circuitry components, where it is desirable to increase the density and reduce the size of such components so that the overall circuitry can be more compact. As the dimensions of IMDs decrease, the electronic circuits of the IMD are formed as integrated circuits to fit within a minimal space. Furthermore, as the dimensions of the components are also being reduced, it is desirable to improve the use of the dimensions within the IMD package.

One response to this desire has been through technological improvements to the packaging for the devices in which the output circuitry is included through such packaging techniques as reconstituted wafer packaging. In particular, development efforts in package-on-package electronic assemblies have focused on producing smaller electronic packages.

### SUMMARY

The techniques of this disclosure generally relate to an electronic assembly and a method of forming such assembly. The assembly can include a first integrated circuit package electrically connected to a second integrated circuit package. In one or more embodiments, the second integrated circuit package can include one or more conductive pillars that extend from a bottom surface of the package. One or more of these conductive pillars can be disposed within a trench formed in a top surface of the first integrated circuit package such that the conductive pillar is electrically connected to a conductor of the first integrated circuit package.

In one example, aspects of this disclosure relate to an electronic assembly that includes a first integrated circuit package having a top surface and a bottom surface. The first integrated circuit package further includes a dielectric layer having a first major surface, a second major surface, and a patterned conductive layer disposed within the dielectric layer; a device disposed on the first major surface of the dielectric layer and electrically connected to the patterned conductive layer; and an encapsulant layer disposed on the device and at least a portion of the first major surface of the dielectric layer. The first integrated circuit package further includes a trench disposed in the top surface of the first integrated circuit package, and a conductor disposed within the trench and electrically connected to the patterned conductive layer of the dielectric layer. The electronic assembly also includes a second integrated circuit package electrically connected to the first integrated circuit package, where the second integrated circuit package includes a top surface and a bottom surface. The bottom surface of the second integrated circuit package faces the top surface of the first integrated circuit package. The second integrated circuit package further includes a dielectric layer having a first major surface, a second major surface, and a patterned conductive layer disposed within the dielectric layer; a device disposed on the first major surface of the dielectric layer and electrically connected to the patterned conductive layer of the dielectric layer; and a conductive pillar that extends from the bottom surface of the second integrated circuit package and is electrically connected to the patterned conductive layer of the second integrated circuit package. The conductive pillar of the second integrated circuit package is disposed within the trench of the first integrated circuit package such that the conductive pillar is electrically connected to the conductor of the first integrated circuit package.

In another example, aspects of this disclosure relate to a method of forming an electronic assembly that includes a first integrated circuit package and a second integrated circuit package electrically connected to the first integrated circuit package. The method includes forming the first integrated circuit package. Forming the first integrated circuit package includes disposing a device on a first major surface of a dielectric layer, where the dielectric layer includes a patterned conductive layer disposed within the dielectric layer. The device is electrically connected to the patterned conductive layer. Forming the first integrated circuit package further includes disposing a conductive pillar on the dielectric layer, where the conductive pillar is electrically connected to the patterned conductive layer; encapsulating the device, the conductive pillar, and at least a portion of the first major surface of the dielectric layer of the first integrated circuit package with an encapsulant; and disposing a trench in the top surface of the integrated circuit package to expose the conductive pillar.

In another example, aspects of this disclosure relate to a method of forming an electronic assembly that includes a first integrated circuit package and a second integrated circuit package electrically connected to the first integrated circuit package. The method includes forming the first integrated circuit package. Forming the first integrated circuit package includes disposing a device on a first major surface of a dielectric layer, where the dielectric layer includes a patterned conductive layer disposed within the dielectric layer. The device is electrically connected to the patterned conductive layer. The method further includes encapsulating the device and at least a portion of the first major surface of the dielectric layer with an encapsulant; disposing a trench between a top surface and a bottom surface of the first integrated circuit package through the encapsulant and the dielectric layer; and disposing a conductor within the trench, where the conductor is electrically connected to the patterned conductive layer of the first dielectric layer.

All headings provided herein are for the convenience of the reader and should not be used to limit the meaning of any text that follows the heading, unless so specified.

The terms “comprises” and variations thereof do not have a limiting meaning where these terms appear in the description and claims. Such terms will be understood to imply the inclusion of a stated step or element or group of steps or elements but not the exclusion of any other step or element or group of steps or elements.

In this application, terms such as “a,” “an,” and “the” are not intended to refer to only a singular entity but include the general class of which a specific example may be used for illustration. The terms “a,” “an,” and “the” are used interchangeably with the term “at least one.” The phrases “at least one of” and “comprises at least one of” followed by a list refers to any one of the items in the list and any combination of two or more items in the list.

The phrases “at least one of” and “comprises at least one of” followed by a list refers to any one of the items in the list and any combination of two or more items in the list.

As used herein, the term “or” is generally employed in its usual sense including “and/or” unless the content clearly dictates otherwise.

The term “and/or” means one or all of the listed elements or a combination of any two or more of the listed elements.

As used herein in connection with a measured quantity, the term “about” refers to that variation in the measured quantity as would be expected by the skilled artisan making the measurement and exercising a level of care commensurate with the objective of the measurement and the precision of the measuring equipment used. Herein, “up to” a number (e.g., up to 50) includes the number (e.g., 50).

Also herein, the recitations of numerical ranges by endpoints include all numbers subsumed within that range as well as the endpoints (e.g., 1 to 5 includes 1, 1.5, 2, 2.75, 3, 3.80, 4, 5, etc.).

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1 is a schematic cross-section view of one embodiment of an electronic assembly.

FIG. 2 is a schematic cross-section view of another embodiment of an electronic assembly.

FIGS. 3A-H are schematic cross-section views of one embodiment of a method of forming the electronic assembly of FIG. 1, where FIG. 3A is a schematic cross-section view of disposing a release layer and a metal layer on a carrier to form an integrated circuit package of the assembly; FIG. 3B is a schematic cross-section view of disposing a dielectric layer and conductors on the carrier; FIG. 3C is a schematic cross-section view of disposing a device on the dielectric layer; FIG. 3D is a schematic cross-section view of disposing an encapsulant layer on the device and one or more portions of a first major surface of the dielectric layer; FIG. 3E is a schematic cross-section view of disposing one or more trenches in a top surface of the integrated circuit package; FIG. 3F is a schematic cross-section view of disposing optional conductive material on a recessed surface of one or more of the trenches; FIG. 3G is a schematic cross-section view of removing the carrier and the metal layer from the integrated circuit package; and FIG. 3H is a schematic cross-section view of disposing one or more conductive pads on a second major surface of the dielectric layer.

FIG. 4 is a schematic cross-section view of another embodiment of an electronic assembly.

FIGS. 5A-H are schematic cross-section views of one embodiment of a method of forming the electronic assembly of FIG. 4, where FIG. 5A is a schematic cross-section view of disposing a release layer and a metal layer on a carrier to form an integrated circuit package of the assembly; FIG. 5B is a schematic cross-section view of disposing a dielectric layer on the carrier; FIG. 5C is a schematic cross-section view of disposing a device on the dielectric layer; FIG. 5D is a schematic cross-section view of disposing an encapsulant layer on the device and one or more portions of a first major surface of the dielectric layer; FIG. 5E is a schematic cross-section view of forming a second dielectric layer on the encapsulant layer; FIG. 5F is a schematic cross-section view of disposing one or more trenches in a top surface of the integrated circuit package; FIG. 5F is a schematic cross-section view of removing the carrier and metal layer from the integrated circuit package; FIG. 5G is a schematic cross-section view of disposing one or more trenches between a top surface and a bottom surface of the integrated circuit package and disposing one or more conductors on sidewalls of one or more trenches; and FIG. 5H is a schematic cross-section view of disposing one or more conductive pads on a second major surface of the dielectric layer.

### DETAILED DESCRIPTION

In general, the present disclosure provides various embodiments of an electronic assembly and a method of forming such assembly. The assembly can include a first integrated circuit package electrically connected to a second integrated circuit package. In one or more embodiments, the second integrated circuit package can include one or more conductive pillars that extend from a bottom surface of the package. One or more of these conductive pillars can be disposed within a trench formed in a top surface of the first integrated circuit package such that the conductive pillar is electrically connected to a conductor of the first integrated circuit package.

In currently-available electronic assemblies, a space between two integrated circuit packages that form the assembly can be relatively large as conductive pads such as solder balls that electrically connect the packages together are disposed between the two packages. Further, foreign material from the packaging process can be entrapped in this space between packages, thereby requiring inspection of the final assembly or an additional step of filling the space to prevent foreign material from creating undesirable current pathways between the two packages.

One or more embodiments of electronic assemblies described herein can provide various advantages over these currently-available assemblies. For example, a distance or gap between a bottom surface of the second integrated circuit package and a top surface of the first integrated circuit package can be minimized. Such minimized distance or gap between packages can help to prevent foreign materials from entering the gap and potentially creating undesirable current pathways between the packages. As a result, additional manufacturing steps such as inspection and gap filling may not be required.

In currently-available electronic assemblies, conductive pillars or conductive pads such as solder balls that are disposed on the second integrated circuit package and electrically connected to the first integrated circuit package can in most cases be no longer than 250 microns to maintain the integrity of the conductive pillar. As a result, a molded integrated circuit package for a die having a thickness greater than 250 μm cannot utilize a conductive pillar. In one or more embodiments of electronic assemblies described herein, a length of the conductive pillar can be reduced as the conductive pillar is disposed within the trench formed in the top surface of the first integrated circuit package. As a result, integrated circuit packages having die that are thicker than 250 μm can utilize conductive pillars for vertical connection of the first and second integrated circuit packages.

One or more embodiments of electronic assemblies described herein can include very small gaps between the packages. Entrapment of foreign material that could otherwise cause unwanted conductive pathways between the first and second integrated circuit packages is minimized by reducing a gap between such packages. As a result, little or no post-production inspection may be required. Further, a total height of the electronic assembly can be reduced.

Microelectronic elements, such as semiconductor chips, are flat bodies with electrical connection contacts disposed on an exterior surface that are connected to the internal electrical circuitry of the element itself. Microelectronic elements are typically packaged to form integrated circuit packages having a surface that is surface mountable with terminals that electrically connect to the element's internal contacts. The package can then be connected to test equipment to determine whether the packaged device conforms to a desired performance standard. Once tested, the package may be connected to a larger circuit, e.g., an electronic assembly utilized in an electronic product such as an implantable medical device.

The various embodiments of electronic assemblies described herein can be utilized in any suitable electronic system. For example, one or more embodiments of electronics assemblies described herein can be utilized in an IMD, ICD, IPG, insertable cardiac monitor, implantable diagnostic monitor, deep brain stimulator, implantable neurostimulator, injectable neurostimulator, implantable ventricular assist device, etc.

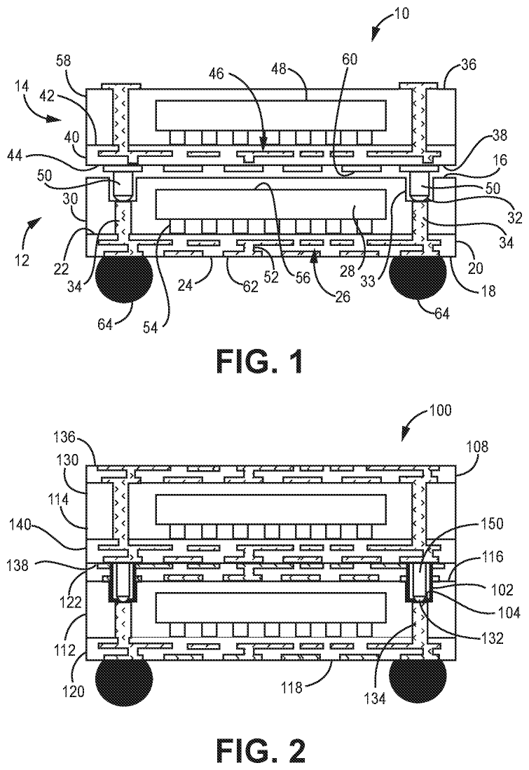

FIG. 1 is a schematic cross-section view of one embodiment of an electronic assembly 10. The assembly 10 includes a first integrated circuit package 12 and a second integrated circuit package 14 electrically connected to the first integrated circuit package. The first integrated circuit package 12 includes a top surface 16 and a bottom surface 18. The first integrated circuit package 12 also includes a dielectric layer 20 that includes a first major surface 22, a second major surface 24, and a patterned conductive layer 26 disposed within the dielectric layer. The first integrated circuit package 12 also includes a device 28 disposed on the first major surface 22 of the dielectric layer 20. Further, the device 28 is electrically connected to the patterned conductive layer 26.

The first integrated circuit package 12 also includes an encapsulant layer 30 disposed on the device 28 and at least a portion of the first major surface 22 of the dielectric layer 20. One or more trenches 32 can be disposed in the top surface 16 of the first integrated circuit package 12. Further, a conductor 34 can be disposed within the trench 32, and the conductor can be electrically connected to the patterned conductive layer 26 of the dielectric layer 20. In one or more embodiments, the trench 32 can be formed first, and the conductor 34 can be disposed within the trench. In one or more embodiments, the conductor 34 can be disposed on the dielectric layer 20, and the encapsulant layer 30 can be disposed on the device 28, at least a portion of the first major surface 22 of the dielectric layer, and the conductor 34. In one or more embodiments, the conductor 34 can be disposed through the encapsulant layer 30.

The electronic assembly 10 can also include the second integrated circuit package 14 electrically connected to the first integrated circuit package 12. The second integrated circuit package 14 includes a top surface 36 and a bottom surface 38, where the bottom surface of the second integrated circuit package faces the top surface 16 of the first integrated circuit package 12. The second integrated circuit package 14 further includes a dielectric layer 40 that includes a first major surface 42, a second major surface 44, and a patterned conductive layer 46 disposed within the dielectric layer. A device 48 of the second integrated circuit package can be disposed on the first major surface 42 of the dielectric layer 40, where the device is electrically connected to the patterned conductive layer 46 of the dielectric layer 40.

The second integrated circuit package 14 also includes one or more conductive pillars 50 that extend from the bottom surface 38 of the second integrated circuit package. The conductive pillars 50 are electrically connected to the patterned conductive layer 46 of the second integrated circuit package 14. At least one conductive pillar 50 can be disposed within a trench 32 of the first integrated circuit package 12 such that the conductive pillar is electrically connected to the conductor 34 of the first integrated circuit package.

The first and second integrated circuit packages, 12, 14 can include any suitable packages, e.g., one or more of fan-out, wire-bond, and lead-frame packages. In one or more embodiments, the first integrated circuit package 12 can be the same as the second integrated circuit package 14. In one or more embodiments, the first integrated circuit package 12 can be different from the second integrated circuit package 14.

Although depicted as including two integrated circuit packages 12, 14, the electronic assembly 10 can include any suitable number of integrated circuit packages. For example, the assembly 10 can include one or more additional integrated circuit packages disposed adjacent the top surface 36 of the second integrated circuit package 14. As used herein, the term “adjacent the top surface” means that an element or component can be disposed closer to the top surfaces 16, 36 of either the first or second integrated circuit package 12, 14 than to the bottom surface 18, 38 of either respective integrated circuit package. In one or more embodiments, one or more additional integrated circuit packages can be disposed adjacent the bottom surface 18 of the first integrated circuit package 12. As used herein, the term “adjacent the bottom surface” means that an element or component can be disposed closed to the bottom surface 18, 38 of either the first or second integrated circuit packages 12, 14 than to the respective top surface 16, 36 of the respective first or second integrated circuit package.

The first and second integrated circuit packages 12, 14 can be spaced apart any suitable distance between the bottom surface 38 of the second integrated circuit package and the top surface 16 of the first integrated circuit package. In one or more embodiments, a distance between the first and second integrated circuit packages can be no greater than 250 μm. In one or more embodiments, one or more portions of the bottom surface 38 of the second integrated circuit package 14 can be in contact with the top surface 16 of the first integrated circuit package 12.

The dielectric layer 20 of the first integrated circuit package 12 includes the first major surface 22 and the second major surface 24, and the patterned conductive layer 26 disposed within the dielectric layer. In one or more embodiments, the second major surface 24 of the dielectric layer 20 defines the bottom surface 18 of the first integrated circuit package 12. In one or more embodiments, one or more additional layers can be disposed on the second major surface 24 of the dielectric layer 20 that would, therefore, define the bottom surface 18 of the first integrated circuit package 12.

The dielectric layer 20 of the first integrated circuit package 12 can include any suitable material or materials. For convenience and without intending to be limiting, the illustration depicts dielectric layer 20 as a monolithic (single) layer. In one or more embodiments, the dielectric layer 20 can include two or more layers or sublayers, and each layer can include the same or different materials. Further, the dielectric layer 20 can have any suitable dimensions. For example, the dielectric layer 20 can have any suitable thickness as measured in a direction orthogonal to the first major surface 22 of the dielectric layer. Further, the dielectric layer 20 can be formed using any suitable technique or techniques.

Disposed within the dielectric layer 20 is the patterned conductive layer 26. The dielectric layer 20 can include any suitable number of patterned conductive layers. In one or more embodiments, the dielectric layer 20 can include a second patterned conductive layer 62 disposed on the second major surface 24 of the dielectric layer. Further, the patterned conductive layers 26, 62 can include any suitable type of conductive layer, e.g., one or more redistribution layers. The patterned conductive layers 26, 62 can be electrically connected to additional patterned conductive layers, devices, conductive pads, etc. using one or more conductive vias 52 that are disposed within the dielectric layer 20. The patterned conductive layers 26, 62 can be formed using any suitable technique or techniques as is further described herein. Further, the patterned conductive layers 26, 62 can be disposed within or on the dielectric layer 20 using any suitable technique or techniques.

The first integrated circuit package 12 can include any suitable dielectric layers. For example, in one or more embodiments, the first integrated circuit package 12 can include a third dielectric layer (not shown) disposed adjacent the top surface 16 of the package between the package and the second integrated circuit package 14.

Disposed on the first major surface 22 of the dielectric layer 20 is the device 28. Although illustrated as including a single device 28, the first integrated circuit package 12 can include any suitable number of devices. The first integrated circuit package 12 can also include any suitable device or devices, e.g., at least one of a capacitor, resistor, passive integrated capacitor system, logic circuit, analog circuit, etc.

The device 28 is electrically connected to the patterned conductive layer 26 of the first integrated circuit package 20 using any suitable technique or techniques. In one or more embodiments, one or more device contacts 54 can be electrically connected to the patterned conductive layer 26 by one or more conductive vias (not shown) that are disposed in the dielectric layer 20. The device contacts 54 can be disposed in any suitable location relative to the patterned conductive layer 26. For example, in one or more embodiments, the device contacts 54 can be disposed between the device 28 and the dielectric layer 20. In one or more embodiments, one or more device contacts 54 can be disposed on a top surface 56 of the device 28 and wire bonded to one or more conductive pads that are electrically connected to the patterned conductive layer 26 by one or more conductive vias.

Disposed on the device 28 and at least a portion of the first major surface 22 of the dielectric layer 20 is the encapsulant layer 30. The encapsulant layer 30 can include any suitable material or materials, e.g., UV curing type or heat curing type, such as BCB, polybenzo-bisoxazole, epoxy, and epoxy resins such as the SINR3170, siloxane resin, manufactured by Shin-Etsu Chemical Co., Ltd., Japan, R4507 EMC (epoxy mold compound) manufactured by Nagase, G730 EMC manufactured by Sumitomo, etc. Further, the encapsulant layer 30 can include any suitable number of layers or sublayers. The encapsulant layer 30 is adapted to encapsulate the device 28. Further, in one or more embodiments, the encapsulant layer 30 is adapted to cover any suitable portion or portions of the first major surface 22 of the dielectric layer 20. In one or more embodiments, the encapsulant layer 30 covers the entire first major surface 22 of the dielectric layer 20. In one or more embodiments, the encapsulant layer 30 is disposed directly onto the first major surface 22 of the dielectric layer 20. In one or more embodiments, one more additional layers can be disposed between the dielectric layer 20 and the encapsulant layer 30. Further, any suitable technique or techniques can be utilized to dispose the encapsulant layer 30 on the device 28 and at least the portion of the first major surface 22 of the dielectric layer 20.

The first integrated circuit package 12 of the electronic assembly 10 also includes the one or more trenches 32 disposed in the top surface 16 of the first integrated circuit package. The first integrated circuit package 12 can include any suitable number of trenches 32. In the embodiment illustrated in FIG. 1, the first integrated circuit package 12 includes two trenches 32. The trenches 32 can be arranged in any suitable location and any suitable pattern or array in the top surface 16 of the first integrated circuit package 12. Each trench 32 can include a recessed surface 33. Further, each trench 32 can take any suitable shape or shapes and have any suitable dimensions. For example, in one or more embodiments, one or more trenches 32 can have a circular cross-section in a plane parallel to the first major surface 16 of the first integrated circuit package 12. Further, each trench 32 can have any suitable depth or height as measured from the first major surface 16 of the first integrated circuit package 12 in a direction orthogonal to such first major surface. In one or more embodiments, at least one trench 32 has a depth of greater than 0 μm and no greater than 250 μm.

The first integrated circuit package 12 can also include one or more conductors 34 that can be disposed within one or more trenches 32. As illustrated in FIG. 1, the first integrated circuit package 12 includes two conductors 34. The conductors 34 can be arranged in any suitable pattern or array within the integrated circuit package 12. Further, the conductors 34 are electrically connected to at least one of patterned conductive layers 26, 62 of the dielectric layer 20. Each conductor 34 can be disposed within the trench 32 and extend any suitable distance from the recessed surface 33 of the trench to at least one of the patterned conductive layers 26, 62. In one or more embodiments, at least one conductor 34 can extend between the recessed surface 33 of at least one trench 32 and the bottom surface 18 of the first integrated circuit package 12.

Each conductor 34 can include any suitable conductor, e.g., a conductive via. Further, each conductor 34 can include any suitable electrically conductive material. The conductors 34 can each take any suitable cross-sectional shape in a plane parallel to the top surface 16 of the first integrated circuit package 12. In one or more embodiments, the conductors 34 have an elliptical (e.g., circular) cross-section in this plane. Further, each conductor 34 can have any suitable width in the plane parallel to the first major surface 16 of the first integrated circuit package 12.

The first integrated circuit package 12 can also include one or more conductive pads 64 disposed on the bottom surface 18 of the first integrated circuit package (or the second major surface 24 of the dielectric layer 20 if such surface provides the bottom surface of the package) using any suitable technique or techniques. One or more conductive pads 64 can be electrically connected to at least one of the second patterned conductive layer 62, the patterned conductive layer 26, and a conductor 34. The conductive pads 64 can include any suitable material or materials and be disposed on the second major surface 24 of the dielectric layer 20 using any suitable technique or techniques.

Electrically connected to the first integrated circuit package 12 is the second integrated circuit package 14. The second integrated circuit package 14 can include any suitable integrated circuit package, e.g., the same integrated circuit packages described regarding the first integrated circuit package 12. All of the design considerations and possibilities regarding the first integrated circuit package 12 apply equally to the second integrated circuit package 14. The second integrated circuit package 14 includes the top surface 36 and the bottom surface 38. The bottom surface 38 faces the top surface 16 of the first integrated circuit package 12.

In the embodiment illustrated in FIG. 1, the second integrated circuit package 14 includes dielectric layer 40 having a first major surface 42 and a second major surface 44. The dielectric layer 40 also includes patterned conductive layer 46 disposed within the dielectric layer. The dielectric layer 40 can include any suitable material or materials, e.g., the same materials described regarding the dielectric layer 20 of the first integrated circuit package 12. Further, the patterned conductive layer 46 of the second integrated circuit package 14 can include any suitable patterned conductive layer or layers, e.g., the same patterned conductive layers 26, 62 described regarding the first integrated circuit package 12. In one or more embodiments, the second integrated circuit package 14 can include a second patterned conductive layer 60 disposed on the second major surface 44 of the dielectric layer 40 of the second integrated circuit package. The second patterned conductive layer 60 can include any suitable material or materials and be disposed in any suitable pattern. In one or more embodiments, the second patterned conductive layer 60 is electrically connected to the patterned conductive layer 46 of the second integrated circuit package 14.

The second integrated circuit package 14 also includes the device 48 disposed on the first major surface 42 of the dielectric layer 40 and electrically connected to the patterned conductive layer 46 of the dielectric layer. The device 48 can include any suitable device or integrated circuit, the e.g., the same devices and circuits described regarding the device 28 of the first integrated circuit package 12. The device 48 can be electrically connected to the patterned conductive layer 46 using any suitable technique or techniques.

In one or more embodiments, the second integrated circuit package 14 can also include an encapsulant layer 58 disposed on the device 48 and at least a portion of the first major surface 42 of the dielectric layer 40. The encapsulant layer 58 can include any suitable encapsulant layer, e.g., encapsulant layer 30 of the first integrated circuit package 12. Further, the encapsulant layer 58 of the second integrated circuit package 14 can be disposed on any suitable portion or portions of the first major surface 42 of the dielectric layer 40.

The second integrated circuit package 14 also includes one or more conductive pillars 50 that extend from the bottom surface 38 of the dielectric layer 40 of the second integrated circuit package. The conductive pillars 50 are electrically connected to at least one of the patterned conductive layers 46, 60 of the second integrated circuit package 14. The conductive pillars 50 can include any suitable electrically conductive material or materials. Further, the conductive pillars 50 can take any suitable cross-sectional shape in a plane parallel to the second major surface 38 of the second integrated circuit package 14. The conductive pillars 50 can each have any suitable length as measured from the bottom surface 38 of the second integrated circuit package 14 in a direction normal to the bottom surface. In one or more embodiments, at least one conductive pillar 50 has a length that is greater than 0 μm and no greater than 250 μm. Although depicted as including two conductive pillars 50, the second integrated circuit package 14 can include any suitable number of conductive pillars.

The conductive pillars 50 can be formed using any suitable technique or techniques. In one or more embodiments, one or more conductive pillars 50 can be plated onto the patterned conductive layer 46 of the dielectric layer 40 using any suitable technique or techniques. In one or more embodiments, one or more conductive pillars 50 can be formed separately and then connected to the patterned conductive layer 46 using any suitable technique or techniques. In one or more embodiments, one or more dielectric sublayers can be disposed on the patterned conductive layer 46 after the conductive pillars 50 have been disposed on the patterned conductive layer.

At least one conductive pillar 50 of the second integrated circuit package 14 is disposed within a trench 32 of the first integrated circuit package 12 such that the conductive pillar is electrically connected to the conductor 34 of the first integrated circuit package associated with the particular trench. By electrically connecting the conductive pillar 50 to the conductor 34, the first integrated circuit package 12 becomes electrically connected to the second integrated circuit package 14.

Any suitable technique or techniques can be utilized to electrically connect the conductive pillar 50 to the conductor 34. For example, the conductive pillar 50 can be bonded to the conductor 34 using any suitable bonding material or materials. In one or more embodiments, a pre-plated solder can be disposed on the conductive pillar 50. In one or more embodiments, solder paste can be disposed within the trench 34 between the conductive pillar 50 and the conductor 34. Further, in one or more embodiments, the conductive pillar 50 of the second integrated circuit package 14 can be dip coated in a solder or conductive epoxy and inserted into the trench 34 such that the conductive epoxy electrically connects and bonds the conductive pillar to the conductor 34 within the trench 32. Further, in one or more embodiments, a conductive epoxy can be dispensed or disposed within the trench 32 such that the conductive epoxy electrically connects and bonds the conductive pillar 50 and the conductor 34.

In one or more embodiments, conductive material can be disposed on one or more portions of a recessed surface of one or more trenches such that a conductive pillar of the second integrated circuit package can be electrically connected to a conductor of the first integrated circuit package via this conductive material. For example, FIG. 2 is a schematic cross-section view of another embodiment an electronic assembly 100. All of the design considerations and possibilities regarding the electronic assembly 10 of FIG. 1 apply equally to the electronic assembly 100 of FIG. 2. Electronic assembly 100 includes a first integrated circuit package 112 and a second integrated circuit package 114 electrically connected to the first integrated circuit package.

One difference between the electronic assembly 100 of FIG. 2 and the electronic assembly 10 of FIG. 1 is that conductive material 102 is disposed on a recessed surface 104 of trench 132 of the first integrated circuit package 112. The conductive material 102 can include any suitable conductive material or materials. Further, any suitable technique or techniques can be utilized to dispose the conductive material 102 within the trench 132. In one or more embodiments, conductive material 102 can be disposed on the entire recessed surface 104 of the trench. In one or more embodiments, the conductive material 102 can be disposed on one or more portions of the recessed surface 104 of the trench 132. The conductive material 102 is electrically connected to conductor 134 of the first integrated circuit package 112. Further, conductive pillar 150 of the second integrated circuit package 114 is disposed within the trench 132 such that the conductive pillar is electrically connected to the conductive material 102 disposed within the trench. In one or more embodiments, the conductive pillar 150 can be electrically connected to both the conductor 134 and the conductive material 102. Any suitable technique or techniques can be utilized to electrically connect the conductive pillar 152 to one or both of the conductive material 102 and the conductor 134. By electrically connecting at least one conductive pillar 150 of the second integrated circuit package 114 to one or both of the conductive material 102 and the conductor 134 of the first integrated circuit package 112, the first integrated circuit package is electrically connected to the second integrated circuit package.

In one or more embodiments, the first integrated circuit package 112 can include a dielectric layer 120 that defines a bottom surface 118 of the first integrated circuit package 112, and a second dielectric layer 122 that defines a top surface 116 of the first integrated circuit package. The first and second dielectric layers 120, 122 can include any suitable dielectric layers, e.g., dielectric layer 20 of integrated circuit package 12 of FIG. 1. In one or more embodiments, at least one of the first and second dielectric layers 120, 122 can include one or more patterned conductive layers (e.g., one or more redistribution layer or layers).

Further, the second integrated circuit package 114 can include a dielectric layer 140 that defines a bottom surface 138 of the package and a second dielectric layer 108 disposed on an encapsulant layer 130 that defines a top surface 136 of the package. The dielectric layers 108, 140 can include any suitable dielectric layer, e.g., dielectric layer 40 of second integrated circuit package 14 of FIG. 1.

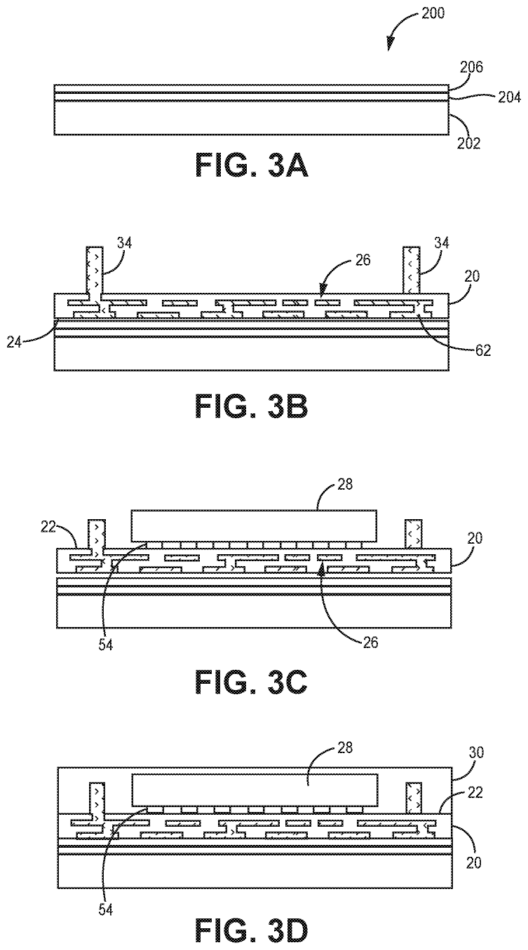

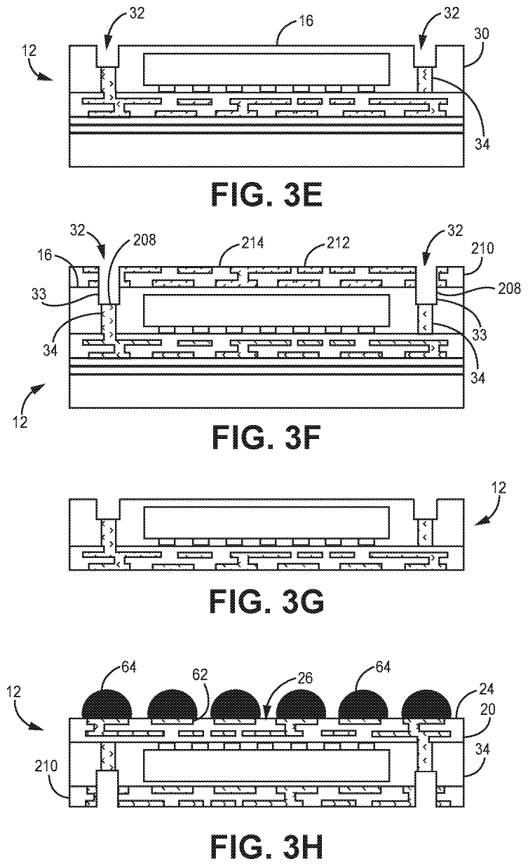

As mentioned herein, any suitable technique or techniques can be utilized to form the electronic assembly 10 of FIG. 1 or electronic assembly 100 of FIG. 2. For example, FIGS. 3A-H are various cross-section views of one embodiment of a method 200 of forming the electronic assembly 10. Although described in reference to the electronic assembly 10 of FIG. 1, the method 200 can be utilized to form any suitable electronic assembly. As shown in FIG. 3A, the first integrated circuit package 12 can be formed utilizing a carrier 202 along with a release layer 204 disposed on the carrier, and a metal layer 206 disposed on the release layer. Any suitable technique or techniques can be utilized to dispose the metal layer 206 onto the release layer 204 and carrier 202. In one or more embodiments, the metal layer 206 can be utilized as a seed layer for electroplating layer 62. After the carrier 202 is removed from the first integrated circuit package 12, the metal layer 206 can also be removed.

As shown in FIG. 3B, the dielectric layer 20 of the first integrated circuit package 12 can be disposed on the metal layer 206 using any suitable technique or techniques. For example, in one or more embodiments, one or more dielectric sublayers can be disposed on the metal layer 206, and the patterned conductive layer 26 can be disposed on one of the dielectric layers using any suitable technique or techniques, e.g., pattern deposition, etching, electroplating, etc. Further, additional patterned conductive layers can be disposed on one or more additional dielectric sublayers to form the dielectric layer 20. For example, second patterned conductive layer 62 can be disposed on the second major surface 24 of the dielectric layer 20 using any suitable technique or techniques. In one or more embodiments, additional dielectric sublayers can be disposed on the patterned conductive layer 26.

As is also shown in FIG. 3B, one or more conductors 34 can be disposed on the patterned conductive layer 26 using any suitable technique or techniques. In one or more embodiments, the conductors 34 can include conductive pillars that are formed, e.g., by plating conductive material onto the patterned conductive layer 26, by attaching conductive pins to the conductive layer, etc. The conductors 34 can be disposed on the patterned conductive layer 26 prior to one or more additional dielectric sublayers being disposed on the patterned conductive layer such that the conductors extend through these additional sublayers as shown in FIG. 3B.

In FIG. 3C, the method 200 includes disposing the device 28 on the first major surface 22 of the dielectric layer 20 using any suitable technique or techniques. The device 28 is disposed on the first major surface 22 of the dielectric layer 20 such that it is electrically connected to the patterned conductive layer 26 via device contacts 54. In one or more embodiments, the device 28 can include a flip chip die that can be connected to the dielectric layer 20 such that is electrically connected to the patterned conductive layer 26. In one or more embodiments, the device 28 can include a wire bond die that is electrically connected to the patterned conductive layer 26 using one or more wire bonds. Although not shown, an additional patterned conductive layer can be disposed on the first major surface 22 of the dielectric layer 20, where the device 28 is electrically connected to this additional patterned conductive layer.

The device 28, the conductors (e.g., conductive pillars) 34, and one or more portions of the first major surface 22 of the dielectric layer 20 of the first integrated circuit package 12 can be encapsulated by disposing the encapsulant layer 30 onto the device, the conductors, and the one or more portions of the first major surface of the dielectric layer using any suitable technique or techniques as shown in FIG. 3D. In one or more embodiments, the encapsulant layer 30 encapsulates the device 28. For example, the encapsulant layer 30 can be compression molded to the device 28 and the first major surface 22 of the dielectric layer 20 using any suitable technique or techniques.

Following encapsulation of the device 28, one or more trenches 32 can be disposed in the top surface 16 of the first integrated circuit package 12 such that one or more conductors 34 are exposed using any suitable technique or techniques, e.g., laser ablation, as shown in FIG. 3E. In one or more embodiments, the trenches 32 can be laser ablated into the encapsulant layer 30 such that the conductors 34 are exposed.

Optional conductive material 208 can be disposed on the recessed surface 33 of one or more trenches 32 such that the conductive material is electrically connected to the conductors 34 of the first integrated circuit package 12 using any suitable technique or techniques as shown in FIG. 3F. Any suitable conductive material can be disposed on one or more portions of the recessed surface 33, e.g., the same conductive materials described regarding conductive material 102 of the electronic assembly 100 of FIG. 2. Further, a second dielectric layer 210 can be disposed on the top surface 16 of the first integrated circuit package 12 using any suitable technique or techniques, e.g., the same techniques utilized to form the dielectric layer 20. The second dielectric layer 210 can include any suitable dielectric layer or layers. In one or more embodiments, the second dielectric layer 210 includes a patterned conductive layer 212 disposed on a first major surface 214 of the second dielectric layer 210. In one or more embodiments, the patterned conductive layer 212 can be electrically connected to the conductive material 208 disposed in one or more trenches 32.

In FIG. 3G, the carrier can be removed from the first integrated circuit package 12 using any suitable technique or techniques. In one or more embodiments, the carrier is debonded from the first integrated circuit package 12. Further, the metal layer 206 can also be removed using any suitable technique or techniques.

As shown in FIG. 3H, one or more conductive pads 64 can be disposed on the second major surface 24 of the dielectric layer 20 using any suitable technique or techniques, e.g., solder ball drop, solder electroplating, solder paste printing followed by reflow, copper pillars with a solder cap, etc. Any suitable conductive pad or pads can be disposed on the second major surface 24 of the dielectric layer 20, e.g., solder ball mounts. The conductive pads 64 can be electrically connected to at least one of the second patterned conductive layer 62, the patterned conductive layer 26, and the conductors 34. In one or more embodiments, one or more conductive pads 64 can be electrically connected to one or more patterned conductive layers of the second dielectric layer 210 of integrated circuit package 12.

Returning to FIG. 1, the second integrated circuit package 14 can be electrically connected to the first integrated circuit package 12 using any suitable technique or techniques. Further, any suitable technique or techniques can be utilized to form the second integrated circuit package 14, e.g., the same techniques utilized to form the first integrated circuit package 12. In one or more embodiments, the second integrated circuit package 14 can be dip coated in flux or conductive epoxy such that conductive pillars 50 are at least partially coated in the flux. The second integrated circuit package 14 can then be disposed adjacent the top surface 16 of the first integrated circuit package 12 using any suitable technique or techniques. In one or more embodiments, the second integrated circuit package 14 can be electrically connected to the first integrated circuit package 12 by inserting the conductive pillars 50 of the second integrated circuit package into one or more trenches 32 of the first integrated circuit package such that the conductive pillars are electrically connected to the conductive pillars or conductors 34 of the first integrated circuit package. In one or more embodiments, the conductive pillars 50 of the second integrated circuit package 14 can also be electrically connected to the optional conductive material 208 (FIG. 3F) disposed on one or more portions of the recessed surfaces 33 of the trenches 32 using any suitable technique or techniques. In one or more embodiments, flux or conductive epoxy disposed on the conductive pillars 50 of the second integrated circuit package 14 can be re-flowed or cured such that the second integrated circuit package remains electrically connected to the first integrated circuit package 12.

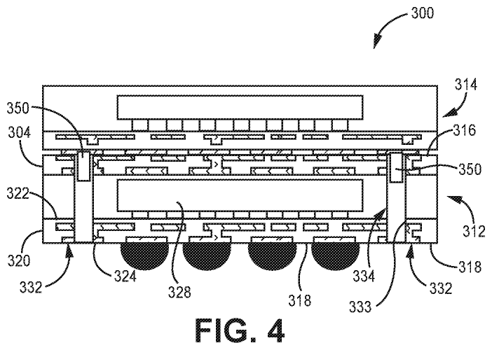

As mentioned herein, one or more integrated circuit packages can be electrically connected together using any suitable technique or techniques. For example, FIG. 4 is a schematic cross-section view of another embodiment of an electronic assembly 300. All of the design considerations and possibilities regarding the electronic assembly 10 of FIG. 1 and the electronic assembly 100 of FIG. 2 apply equally to the electronic assembly 300 of FIG. 4. As illustrated in FIG. 4, the electronic assembly 300 includes a first integrated circuit package 312 electrically connected to a second integrated circuit package 314. The first integrated circuit package 312 includes one or more trenches 332 disposed between a top surface 316 and a bottom surface 318 of the first integrated circuit package. Any suitable technique or techniques can be utilized to dispose trenches 332 through the first integrated circuit package 312, e.g., laser ablation, drilling, etc. One or more conductors 334 can be disposed within the trench 332 using any suitable technique or techniques. In one or more embodiments, conductive material can be disposed on one or more portions of a sidewall 333 of one or more trenches 332 to provide conductors 334. In one or more embodiments, conductive material can be disposed in the trenches 332 by plating the sidewalls 333 of the trenches to form conductors 334. The conductors 334 can be electrically connected to one or more patterned conductive layers disposed on or in dielectric layer 320 and second dielectric layer 304.

The first integrated circuit package 312 is electrically connected to the second integrated circuit package 314 using any suitable technique or techniques. In one or more embodiments, conductive pillars 350 of the second integrated circuit package 314 are disposed within trenches 332 of the first integrated circuit package 312 such that the conductive pillars are electrically connected to the conductors 334 disposed within the trenches. In one or more embodiments, a pre-plated solder can be disposed on side surfaces of the conductive pillars 350 of the second integrated circuit package 314 to electrically connect the pillars to the conductors 334 of the first integrated circuit package 312. In one or more embodiments, preformed solder can be disposed around the trenches 334 on the top surface 316 of the first integrated circuit package 312, and the conductive pillars 350 of the second integrated circuit package 314 can be coated by the preformed solder as the pillars are inserted into the trenches 332. Further, in one or more embodiments, solder paste can be disposed (e.g., by printing) around trenches 332 on the top surface 316 of the first integrated circuit package 312 such that the conductive pillars 350 become coated with the solder paste as the pillars are inserted into the trenches. Further, in one or more embodiments, the pillars 350 of the second integrated circuit package 314 can be dip coated in flux or conductive epoxy prior to insertion of the pillars into the trenches 332. In one or more embodiments, flux or conductive epoxy can be disposed into the trenches 332 from the bottom surface 318 of the first integrated circuit package 312 after the conductive pillars 350 of the second integrated circuit package 314 are disposed within the trenches 332. Any suitable technique or techniques can be utilized to dispose the solder into the trenches 332 from the bottom surface 318 of the first integrated circuit package 312.

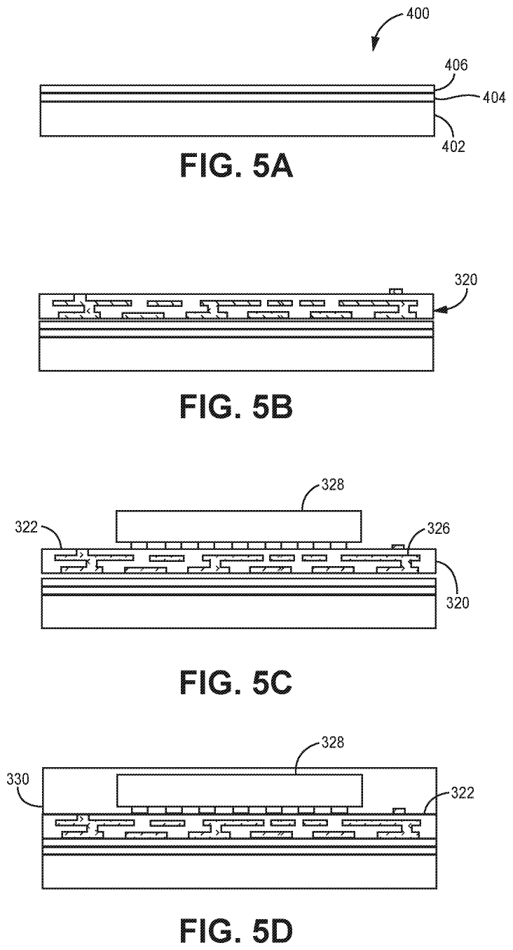

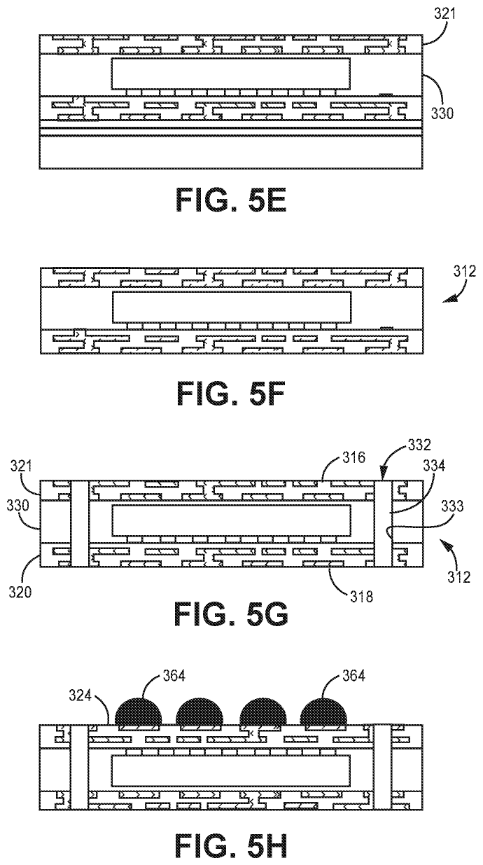

Any suitable technique or techniques can be utilized to form the electronic assembly 300 of FIG. 4. For example, FIGS. 5A-H are various cross-section views of a method 400 of forming the integrated circuit package 300 of FIG. 4. Although described in reference to the electronic assembly 300, the method 500 can be utilized to form any suitable electronic assembly. As shown in FIG. 5A, a metal layer 406 can be disposed on a carrier 402 using any suitable technique or techniques. In one or more embodiments, a release layer 404 can be disposed between the metal layer 406 and the carrier 402. The dielectric layer 320 of first integrated circuit package 312 can be disposed on the metal layer 406 using any suitable technique or techniques as shown in FIG. 5B. The device 328 can be disposed on the first major surface 322 of the dielectric layer 320 as shown in FIG. 5C using any suitable technique or techniques. The device 328 can be electrically connected to the patterned conductive layer 326 using any suitable technique or techniques. As shown in FIG. 5D, the device 328 can be encapsulated along with one or more portions of the first major surface 322 of the dielectric layer 320 using any suitable encapsulant to provide encapsulant layer 330. In one or more embodiments, the second dielectric layer 304 can be disposed on the encapsulant layer 330 using any suitable technique or techniques as shown in FIG. 5E.

In FIG. 5F, the carrier 400 and the metal layer 406 can be removed from the first integrated circuit package 312 using any suitable technique or techniques. Further as shown in FIG. 5G, trenches 332 can be disposed between the top surface 316 and the bottom surface 318 of the first integrated circuit package 312 using any suitable technique or techniques. The trenches 332 can extend through the dielectric layer 320, the second dielectric layer 304, and the encapsulant layer 330. Further, one or more conductors 334 can be disposed on sidewalls 333 of one or more trenches 332 using any suitable technique or techniques.

As shown in FIG. 5H, one or more conductive pads 364 can be disposed on the second major surface 324 of the dielectric layer 320 using any suitable technique or techniques, e.g., solder ball drop, solder electroplating, solder paste printing followed by reflow, copper pillar with solder cap, etc. Any suitable conductive pad or pads 364 can be disposed on the second major surface 324 of the dielectric layer 320, e.g., solder balls. The conductive pads 364 can be electrically connected to one or more patterned conductive layers of at least one of the dielectric layer 320 and second dielectric layer 304 using any suitable technique or techniques.

The second integrated circuit package 314 can be formed using any suitable technique or techniques, e.g., the same techniques described regarding second integrated circuit package 14 of the electronic assembly 10 of FIG. 1. The second integrated circuit package 314 can be electrically connected to the first integrated circuit package 312 as shown in FIG. 4 using any suitable technique or techniques. For example, the second integrated circuit package 314 can be dip coated in flux or conductive epoxy such that conductive pillars 350 are at least partially coated in the flux. The second integrated circuit package 314 can be electrically connected to the first integrated circuit package 312 by inserting the conductive pillars 350 of the second integrated circuit package into corresponding trenches 332 of the first integrated circuit package such that the conductive pillars are electrically connected to the conductors 334 of the first integrated circuit package. In one or more embodiments, flux or conductive epoxy disposed on the conductive pillars 350 of the second integrated circuit package 314 can be re-flowed or cured such that the second integrated circuit package remains electrically connected to the first integrated circuit package 312.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include computer-readable storage media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

What is claimed is:

1. An electronic assembly comprising:
a first integrated circuit package comprising a top surface and a bottom surface, wherein the first integrated circuit package further comprises:
a dielectric layer comprising a first major surface, a second major surface, and a patterned conductive layer disposed within the dielectric layer;
a device disposed on the first major surface of the dielectric layer and electrically connected to the patterned conductive layer;
an encapsulant layer disposed on the device and at least a portion of the first major surface of the dielectric layer;
a trench disposed in the top surface of the first integrated circuit package; and
a conductor disposed within the trench and electrically connected to the patterned conductive layer of the dielectric layer; and
a second integrated circuit package electrically connected to the first integrated circuit package and comprising a top surface and a bottom surface, wherein the bottom surface of the second integrated circuit package faces the top surface of the first integrated circuit package, wherein the second integrated circuit package further comprises:
a dielectric layer comprising a first major surface, a second major surface, and a patterned conductive layer disposed within the dielectric layer;
a device disposed on the first major surface of the dielectric layer and electrically connected to the patterned conductive layer of the dielectric layer; and
a conductive pillar that extends from the bottom surface of the second integrated circuit package and is electrically connected to the patterned conductive layer of the second integrated circuit package, wherein the conductive pillar of the second integrated circuit package is disposed within the trench of the first integrated circuit package such that the conductive pillar is electrically connected to the conductor of the first integrated circuit package.

2. The assembly of claim 1, wherein the conductor of the first integrated circuit package comprises a conductive via that extends between the trench and the patterned conductive layer of the first integrated circuit package.

3. The assembly of claim 1, wherein the first integrated circuit package further comprises a conductive material disposed on a recessed surface of the trench that is electrically connected to the conductor, wherein the conductive pillar of the second integrated circuit package is electrically connected to the conductive material disposed within the trench.

4. The assembly of claim 1, wherein at least a portion of the bottom surface of the second integrated circuit package is in contact with the top surface of the first integrated circuit package.

5. The assembly of claim 1, further comprising a second dielectric layer disposed on the encapsulant layer of the first integrated circuit package such that the device of the first integrated circuit package is between the dielectric layer and the second dielectric layer.

6. The assembly of claim 1, further comprising a second patterned conductive layer disposed on the second major surface of the dielectric layer of the second integrated circuit package.

7. The assembly of claim 1, wherein the conductive pillar of the second integrated circuit package has a length measured from the second major surface of the dielectric layer of the second integrated circuit package of greater than 0 μm and no greater than 250 μm.

8. The assembly of claim 1, wherein the trench extends between the top and bottom surfaces of the first integrated circuit package, wherein the conductor comprises conductive material disposed on a sidewall of the trench and electrically connected to the patterned conductive layer of the first integrated circuit package.

9. A method of forming an electronic assembly comprising a first integrated circuit package and a second integrated circuit package electrically connected to the first integrated circuit package, the method comprising forming the first integrated circuit package, wherein forming the first integrated circuit package comprises:
disposing a device on a first major surface of a dielectric layer, wherein the dielectric layer comprises a patterned conductive layer disposed within the dielectric layer, wherein the device is electrically connected to the patterned conductive layer;
disposing a conductive pillar on the dielectric layer, wherein the conductive pillar is electrically connected to the patterned conductive layer;
encapsulating the device, the conductive pillar, and at least a portion of the first major surface of the dielectric layer of the first integrated circuit package with an encapsulant; and
disposing a trench in the top surface of the integrated circuit package to expose the conductive pillar.

10. The method of claim 9, further comprising disposing conductive material on a recessed surface of the trench that is electrically connected to the conductive pillar of the first integrated circuit package.

11. The method of claim 9, wherein disposing the trench in the top surface of the first integrated circuit package comprises ablating the encapsulant layer of the first integrated circuit package to expose the conductive pillar.

12. The method of claim 9, further comprising disposing a conductive pad on a bottom surface of the first integrated circuit package, wherein the conductive pad is electrically connected to the patterned conductive layer of the first integrated circuit package.

13. The method of claim 9, wherein disposing the conductive pillar on the dielectric layer of the first integrated circuit package comprises electroplating the conductive pillar onto the dielectric layer.

14. The method of claim 9, further comprising forming the second integrated circuit package, wherein forming the second integrated circuit package comprises:
disposing a device on a first major surface of a dielectric layer, wherein the dielectric layer comprises a patterned conductive layer disposed within the dielectric layer, wherein the device is electrically connected to the patterned conductive layer; and
disposing a conductive pillar on a second major surface of the dielectric layer such that it extends from a bottom surface of the second integrated circuit package, wherein the conductive pillar is electrically connected to the patterned conductive layer.

15. The method of claim 14, further comprising electrically connecting the second integrated circuit package to the first integrated circuit package.

16. The method of claim 15, wherein electrically connecting the second integrated circuit package to the first integrated circuit package comprises inserting the conductive pillar of the second integrated circuit package into the trench of the first integrated circuit package such that the conductive pillar of the second integrated circuit package is electrically connected to the conductive pillar of the first integrated circuit package.

17. A method of forming an electronic assembly comprising a first integrated circuit package and a second integrated circuit package electrically connected to the first integrated circuit package, the method comprising forming the first integrated circuit package, wherein forming the first integrated circuit package comprises:
disposing a device on a first major surface of a dielectric layer, wherein the dielectric layer comprises a patterned conductive layer disposed within the dielectric layer, wherein the device is electrically connected to the patterned conductive layer;
encapsulating the device and at least a portion of the first major surface of the dielectric layer with an encapsulant;
disposing a trench between a top surface and a bottom surface of the first integrated circuit package through the encapsulant and the dielectric layer; and
disposing a conductor within the trench, wherein the conductor is electrically connected to the patterned conductive layer of the first dielectric layer.

18. The method of claim 17, wherein disposing the conductor within the trench comprises disposing a conductive material on a sidewall of the trench.

19. The method of claim 17, further comprising forming a second patterned conductive layer on a second major surface of the dielectric layer of the first integrated circuit package to form the bottom surface of the first integrated circuit package, wherein the trench is disposed through the dielectric layer.

20. The method of claim 17, further comprising disposing a conductive pad on the bottom surface of the first integrated circuit package that is electrically connected to the patterned conductive layer of the first integrated circuit package.

21. The method of claim 17, further comprising forming the second integrated circuit package, wherein forming the second integrated circuit package comprises:
disposing a device on a first major surface a dielectric layer, wherein the dielectric layer comprises a patterned conductive layer disposed within the dielectric layer, wherein the device is electrically connected to the patterned conductive layer; and
disposing a conductive pillar on a second major surface of the dielectric layer such that it extends from a bottom surface of the second integrated circuit package, wherein the conductive pillar is electrically connected to the patterned conductive layer of the second integrated circuit package.

22. The method of claim 21, further comprising electrically connecting the second integrated circuit package to the first integrated circuit package.

23. The method of claim 22, wherein electrically connecting the second integrated circuit package to the first integrated circuit package comprises inserting the conductive pillar of the second integrated circuit package into the trench of the first integrated circuit package such that the conductive pillar of the second integrated circuit package is electrically connected to the conductor of the first integrated circuit package.
